(19) United States
(12) Patent Application Publication
(10) Pub. No.: US 2026/0022086 A1
(43) Pub. Date: Jan. 22, 2026

(54) PROCESSES FOR MAKING ETHYLENE DICHLORIDE FROM MONOETHYLENE GLYCOL

(71) Applicant: New Product Innovation, LLC, Johnston, IA (US)

(72) Inventor: David James Schreck, Lake City, MN (US)

(73) Assignee: New Product Innovation, LLC, Johnston, IA (US)

(21) Appl. No.: 19/276,839

(22) Filed: Jul. 22, 2025

Related U.S. Application Data
(60) Provisional application No. 63/674,028, filed on Jul. 22, 2024.

(51) Int. Cl. C07C 17/16; C07C 17/386

(52) U.S. Cl. CPC C07C 17/16; C07C 17/386

(57) ABSTRACT

A process is disclosed for producing ethylene dichloride from monoethylene glycol and hydrogen chloride in the presence of water, thereby forming an ethylene dichloride-rich liquid phase that can be readily separated from a coexisting aqueous phase. The reaction is carried out under conditions that limit both 2-chloroethanol and ethylene dichloride in the vapor phase, facilitating high conversion efficiencies and minimizing ethylene dichloride losses. The process includes a reaction step in which monoethylene glycol is converted to 2-chloroethanol and subsequently to ethylene dichloride, generating water that aids in separating by-products from the ethylene dichloride-rich phase. A phase-separation system is employed to decant the heavier, ethylene dichloride phase from an aqueous phase containing residual reactants and by-products. Additional purification steps, such as washing with substantially anhydrous monoethylene glycol, further remove water, acids, and 2-chloroethanol, producing high-purity ethylene dichloride. Recycling unconverted monoethylene glycol and 2-chloroethanol to the reactor increases overall conversion and enhances process economics.

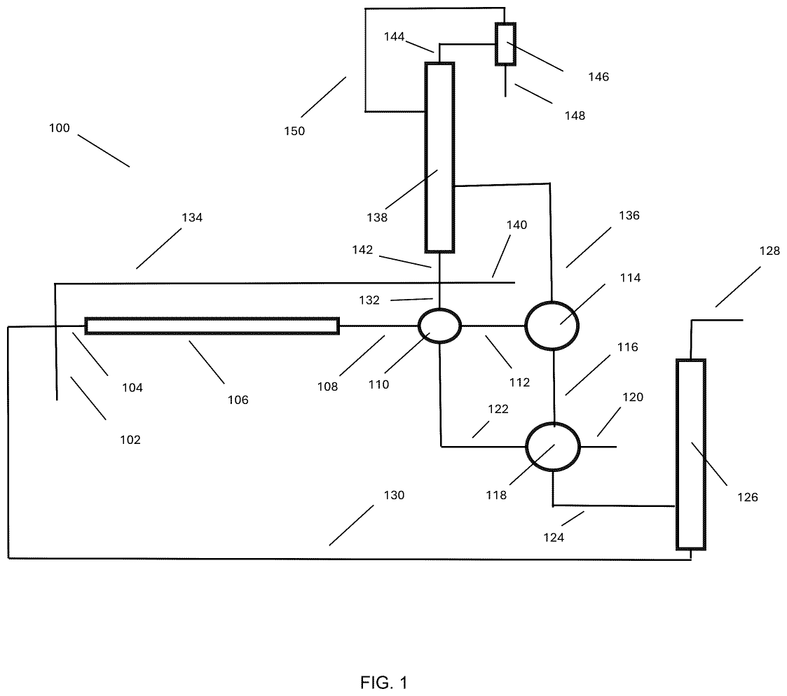

### CROSS-REFERENCE TO RELATED APPLICATION(S)

This application claims priority to U.S. Provisional Application No. 63/674,028 filed Jul. 22, 2024, and entitled “PROCESSES FOR MAKING ETHYLENE DICHLORIDE FROM MONOETHYLENE GLYCOL,” which is hereby incorporated by reference in its entirety under 35 U.S.C. § 119 (e).

### TECHNICAL FIELD

This invention pertains to processes for making ethylene dichloride from monoethylene glycol, and particularly process having enhanced commercial viability.

### BACKGROUND

Ethylene dichloride (1,2dichloroethane) is a commodity chemical having commercial applications ranging from being a solvent to being an intermediate for organic chemicals and polyvinyl chloride. Ethylene dichloride is typically made by the catalyzed reaction of ethylene with chlorine or the oxychlorination of ethylene. A general interest exists in making chemicals from sustainable resources, including making ethylene dichloride.

Proposals have been made for the conversion of multihydroxylated-aliphatic hydrocarbons or esters to chlorohydrin. See, for instance, Kruper, et al., U.S. Pat. No. 8,088,957 B2. Kruper, et al., in Example 43, disclose making chloroethanol among other products from a feed comprising ethylene glycol and 1,2-propylene glycol. The patentees made no mention in that example of whether or not ethylene dichloride was coproduced.

Proposals have also been made to manufacture ethylene glycol from renewable resources such as carbohydrates. These alternative processes include catalytic routes such as hydrogenolysis of sugar and a two-catalyst process using a retro-aldol catalyst to #4714069 1 generate intermediates from sugar that can be hydrogenated over a hydrogenation catalyst to produce ethylene glycol and propylene glycol.

Accordingly, processes for making ethylene dichloride from monoethylene glycol are of interest to provide an alternative route to ethylene dichloride, which route would be able, if desired, to provide ethylene dichloride from sustainable resources. However, to be of commercial acceptability, such processes would have to achieve high selectivity to ethylene dichloride and be energy efficient.

### BRIEF SUMMARY

Ethylene dichloride (1,2dichloroethane) is a commodity chemical having commercial applications ranging from being a solvent to being an intermediate for organic chemicals and polyvinyl chloride. Ethylene dichloride is typically made by the catalyzed reaction of ethylene with chlorine or the oxychlorination of ethylene. A general interest exists in making chemicals from sustainable resources, including making ethylene dichloride.

Proposals have been made for the conversion of multihydroxylated-aliphatic hydrocarbons or esters to chlorohydrin. See, for instance, Kruper, et al., U.S. Pat. No. 8,088,957 B2. Kruper, et al., in Example 43, disclose making chloroethanol among other products from a feed comprising ethylene glycol and 1,2-propylene glycol. The patentees made no mention in that example of whether or not ethylene dichloride was co-produced.

Proposals have also been made to manufacture ethylene glycol from renewable resources such as carbohydrates. These alternative processes include catalytic routes such as hydrogenolysis of sugar and a two-catalyst process using a retro-aldol catalyst to generate intermediates from sugar that can be hydrogenated over a hydrogenation catalyst to produce ethylene glycol and propylene glycol.

Accordingly, processes for making ethylene dichloride from monoethylene glycol are of interest to provide an alternative route to ethylene dichloride, which route would be able, if desired, to provide ethylene dichloride from sustainable resources. However, to be of commercial acceptability, such processes would have to achieve high selectivity to ethylene dichloride and be energy efficient.

### SUMMARY

By this invention processes are provided for making ethylene dichloride from monoethylene glycol and hydrogen chloride in the presence of water. Ethylene dichloride is recovered from a reaction mixture also containing at least 2—chloroethanol, hydrogen chloride and water. The recovery is under conditions where two liquid phases are formed, an ethylene dichloride-rich liquid phase and an aqueous phase, and an ethylene dichloride product is recovered by phase separation. While not wishing to be limited by theory, it is believed that the presence of substantial amounts of water in the aqueous phase as well as the polarity of the aqueous phase, provide an ethylene dichloride-rich liquid phase that can be readily processed to make high purity ethylene dichloride.

In a broad aspect, the process of this invention making ethylene dichloride from monoethylene glycol and hydrochloric acid in the presence of water comprise:

a) contacting in a reaction zone monoethylene glycol and hydrochloric acid at a temperature and for a time sufficient to convert at least about 25, preferably at least about 35, and sometimes at least about 50 percent, of the monoethylene glycol to ethylene dichloride, to provide a reaction product comprising a liquid phase product containing ethylene dichloride, water, hydrochloric acid, and 2-choroethanol and optionally a gas phase comprising azeotrope of ethylene dichloride and water, wherein
i. the temperature and pressure of the reaction is sufficient to provide a ratio of 2-chloroethanol in azeotrope with water in the gas phase to 2-chloroethanol in the liquid phase of less than 1:10,
ii. sufficient hydrogen chloride is present such that the pH of the reaction product is at a pH of below about 2, and
iii. the reaction product comprises between about 20 and 80 mass percent water;

b) providing the reaction product (i) at a temperature and pressure sufficient to provide a ratio of ethylene dichloride as an azeotropic mixture with water in the gas phase to ethylene dichloride in liquid phase of less than 1:10, and (ii) under conditions sufficient to form a denser, liquid phase rich in ethylene dichloride and a less dense liquid aqueous phase comprising water, hydrochloric acid, and 2-chloroethanol; and

c) selectively withdrawing from step (b) at least a portion of the heavier, liquid phase as an ethylene dichloride product.

The processes of this invention can be batch, semi-batch and continuous. For the purposes herein, the conversion of monoethylene glycol is based upon the amount of monoethylene glycol fed to the reaction zone excluding the amount of monoethylene glycol recycled to the reaction zone. The reaction of hydrogen chloride with monoethylene glycol to for 2-chloroethanol and water proceeds more rapidly than the reaction of 2-chloroethanol with hydrogen chloride to ethylene dichloride and water. Consequently, the reaction product from step (a) contains 2-chloroethanol, which can be recycled to the reaction zone, thereby increasing the conversion of the monoethylene glycol. Typically, the reaction product also contains unreacted monoethylene glycol. Thus, the conversion to ethylene dichloride would not be limited by monoethylene glycol availability, but rather by reaction kinetics. In some instances, the monoethylene glycol comprises at least about 5, say 5 to 20 or 25, mass percent of the reaction product. By recycling the monoethylene glycol and 2-chloroethanol to the reaction zone, essentially complete conversion of the exogenous monoethylene glycol added to the reaction zone can be achieved. The water in the reaction product of step (a) can be that formed by the reaction occurring in the reaction zone plus any additional water added to the reaction zone or, in a continuous mode of operation, recycled to the reaction zone. Thus use of added or recycled water is often desired to enhance the separation of 2-chloroethnaol and hydrogen chloride from the ethylene dichloride-rich phase in step (b). The pressure and temperature of step (a) can be sufficient to maintain substantially all the ethylene dichloride in a liquid phase, or a portion or all of the ethylene dichloride can exist in the vapor phase as an azeotrope with water. Where all or a portion of the ethylene dichloride is in the vapor phase in the reaction zone, preferably the azeotrope of ethylene dichloride and water is contacted with the liquid phase in step (b) and the ratio of ethylene dichloride as an azeotropic mixture with water in the gas phase to ethylene dichloride in liquid phase of less than 1:10. Steps (a) and (b) can be conduced in the same vessel or a reaction product from step (a) can be passed to a different vessel maintained under different conditions than those in step (a), e.g., under conditions sufficient to condense any azeotrope of ethylene dichloride and water. In some instances, step (b) is conducted at a temperature below the boiling point at the pressure of step (b) of the ethylene dichloride and water azeotrope.

In a preferred embodiment, the ethylene dichloride product of step (c) is contacted with substantially anhydrous monoethylene glycol whereby residual water, 2-chloroethanol and hydrochloric acid contained in the ethylene dichloride is stripped to the monoethylene glycol which is a less dense liquid phase than the denser, ethylene dichloride phase, and at least an aliquot portion is selectively separated from the ethylene dichloride phase to provide a purified ethylene dichloride product.

In another preferred embodiment, at least an aliquot portion of the less dense liquid phase of step (b) is withdrawn from the decanting zone and is subjected to azeotropic distillation to provide an overhead comprising water and a bottoms fraction comprising 2-chloroethanol, water and hydrochloric acid, and at least a portion of the bottoms fraction is passed to the reaction zone of step (a). Thus, in a continuous process, the water concentration in the reaction product of step (a) can be maintained at a desired level and 2-choroethanol is returned to step (a) thereby enhancing the selectivity of conversion to ethylene dichloride. Often, at least about 85, preferably at least about 90, percent of the monoethylene glycol can be converted to ethylene dichloride.

In yet another preferred embodiment of this invention, the reaction product of step (a) is cooled by indirect heat exchange by at least a portion of the monoethylene glycol for step (a).

Preferably the processes of this invention are conducted on a continuous basis.

### BRIEF DESCRIPTION OF THE DRAWINGS

The drawing is a schematic depiction of an apparatus that can be used to practice the processes of this invention.

### DETAILED DESCRIPTION

All patents, published patent applications and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3. 6.3, 0.815 and so forth) are included as are all intermediate ranges (e.g., 0.2-0.5, 0.54-0.913, and so forth).

The use of the terms “a” and “an” is intended to include one or more of the element described.

Aqueous and aqueous solution mean that water is present but does not require that water be the predominant component. For purposes of illustration and not in limitation, a solution of 90 volume percent of ethylene glycol and 10 volume percent water would be an aqueous solution. Aqueous solutions include liquid media containing dissolved or dispersed components such as, but not in limitation, colloidal suspensions and slurries.

Bio-sourced carbohydrate feedstock means a product that includes carbohydrates sourced, derived or synthesized from, in whole or in significant part, to biological products or renewable agricultural materials (including, but not limited to, plant, animal and marine materials) or forestry materials.

High shear mixing involves providing a fluid traveling at a different velocity relative to an adjacent area which can be achieved through stationary or moving mechanical means to effect a shear to promote mixing. As used herein, the components being subjected to high shear mixing may be immiscible, partially immiscible or miscible.

pH of an aqueous solution is determined at ambient pressure and temperature. In determining the pH of, for example the aqueous, hydrogenation medium or the product solution, the liquid is cooled and allowed to reside at ambient pressure and temperature for 2 hours before determination of the pH.

A reactor can be one or more vessels in series or in parallel and a vessel can contain one or more zones. A reactor can be of any suitable design for continuous operation including, but not limited to, tanks and pipe or tubular reactor and can have, if desired, fluid mixing capabilities. Types of reactors include, but are not limited to, laminar flow reactors, fixed bed reactors, slurry reactors, fluidized bed reactors, moving bed reactors, simulated moving bed reactors, trickle-bed reactors, bubble column reactors, cavitation reactors and loop reactors.

The invention will be further described in connection with the drawing. The drawing is a schematic description of an apparatus, generally designated as 100, useful in conducting the processes of this invention and is not intended to be in limitation of the invention. The drawing omits minor equipment such as pumps, compressors, valves, instruments and other devices the placement of which and operation thereof are well known to those practiced in chemical engineering. The drawing also omits ancillary unit operations.

The processes of this invention use hydrogen chloride and monoethylene glycol as the reactants to make ethylene dichloride. The monoethylene glycol may be from any convenient source including the existing commercial processes involving the catalytic epoxidation of ethylene followed by reaction with water and processes for making monoethylene glycol from bio-sourced carbohydrate feed stock, such as sugars. The hydrogen chloride can be anhydrous, or preferably contains water, e.g., often the mass ratio of water to total hydrogen chloride and water in the feed to the reactor is in the range of about 0.05:1 to 0.7:1, say, about 0.1:1 to 0.5:1. The processes of this invention require the presence of water. As the reaction of hydrogen chloride and monoethylene glycol generates water, all or a portion, of the necessary water can be provided by the reaction itself. In a preferred embodiment the hydrogen chloride can be supplied as commercial, aqueous hydrogen chloride which is nominally 37 percent water.

As shown, the hydrogen chloride is supplied to the apparatus via line 102 and is admixed with monoethylene glycol, the source of which will be described later, and the admixed reactants are passed via line 104 to reactor 106. The molar ratio of hydrogen chloride to monoethylene glycol supplied to reactor 106 is at least about 1.5:1, and is often in the range of 1.75:1 to 5:1. Preferably, sufficient hydrogen chloride is supplied to reactor 106 that the reactor effluent has a pH of less than about 2, and in some instances, less than about 1.5.

The fluid in reactor 106 is maintained at conditions sufficient to result in the production of ethylene dichloride and to substantially maintain the azeotrope of 2-chloroethanol and water in the liquid phase. Often the conditions are such that the mole ratio of (i) the azeotrope of 2-chloroethanol and water to (ii) 2-chloroethanol in the liquid phase is less than about 1:10, and preferably less than about 1:20. The reaction temperature is typically greater than about 70° C., and often above about 100° C., say, 100° C. to 200°° C., and frequently between about 110° C. and 170° C. As the 2-chloroethanol azeotrope has a normal boiling point of about 98° C., the reaction is generally conducted under superatmospheric pressure sufficient to maintain the desired fraction of 2-chloroethanol in the liquid phase. For a given reaction temperature, those skilled in the art can readily ascertain the pressures required to maintain the desired mole fraction of the 2-chloroethanol in the liquid phase. The pressure is typically in the range of 100 to 10,000 kPa absolute.

The product, ethylene dichloride, also forms an azeotrope with water that has a normal boiling point of about 72° C. In one embodiment the pressure of the reaction can be such that the mole ratio of (i) ethylene dichloride as an azeotropic mixture with water in the gas phase to (ii) ethylene dichloride in liquid phase of less than 1:10. In an alternative embodiment, the reactor pressure is insufficient to maintain the ethylene dichloride and water azeotrope in the liquid phase. In which case, at least a portion of the ethylene dichloride and water azeotrope can be withdrawn from the reactor as a vapor phase and/or the reactor can be operated with a mixed vapor and liquid phase. For example, a tubular reactor can be operated in a continuous process using mixed vapor and liquid phases.

The reaction can be conducted in the presence of catalyst. Any suitable catalyst can be used. Catalysts known in the art include carboxylic acids, carboxylic anhydrides, carboxylic chlorides, carboxylic esters, lactones, lactams, amides and carboxylic salts, e.g., water soluble salts of, say, sodium, potassium, magnesium, calcium, and the like. See, for instance, U.S. Pat. No. 8,088,957 B2, column 8, line 65 through column 10, line 67,herein incorporated by reference. The catalyst is used in a catalytically effective amount, and the amount used will depend upon the activity of the catalyst and the desired quantum of activity. Usually, the mass ratio of catalyst to monoethylene glycol being supplied is in the range of about 0.01:1 to 1:1.

The duration of the reaction is sufficient to convert at least 50, preferably 60 to 99, and sometimes between about 70 and 95, percent of the monoethylene glycol to ethylene dichloride. As known in the art, conditions such as temperature, catalyst and amount, and mixing of reactants will be determinate of the duration of the reaction.

Reactor 106 can be operated in batch, semi-batch, and preferably, continuous, mode.

Reactor 106 can be of any suitable design. Tubular reactors provide the advantage described above for continuous operations. Tank, and especially stirred tank, reactors can find application for batch, semi-batch and continuous operations.

A reaction product is withdrawn from reactor 106 via line 108 and is passed to heat exchanger 110. Line 108 may be a single line or a plurality of lines especially where a vapor containing the azeotrope of ethylene dichloride and water is separately withdrawn from reactor 106. The reaction product comprises a liquid phase product containing ethylene dichloride, water, hydrochloric acid, 2-choroethanol and sometimes monoethylene glycol and optionally a gas phase comprising azeotrope of ethylene dichloride and water. Depending upon the temperature and pressure of the product, other azeotropes such as that of 2-chloroethanol and water and of hydrochloric acid and water, may also be present. For reasons that will be discussed later, a substantial portion of the reaction product comprises water, and the reaction product has a pH of less than about 2. The portion of water will depend upon the amount of water fed to rector 106 as well as the water produced during the reaction. In general, the reaction product comprises between about 20 and 80, say, about 30 to 60, mass percent water.

In heat exchanger 110, the reaction product is cooled and then passed via line 112 to decanter 114. The cooling is to a temperature where, in the decanter, substantially all the ethylene dichloride is in the liquid phase and less than about 5, preferably less than about 2, and most preferably less than about 0.5, percent of the ethylene dichloride is in the vapor phase as an azeotrope with water. As the ethylene dichloride and water azeotrope boiling point is lower than that of the 2-choroethanol and water azeotrope, essentially all the 2-chloroethanol is in the liquid phase of the reaction product. The pressure in decanter 114 can be substantially the same as that in reactor 106, or, if desired, at a lower pressure, e.g., between about 100 to 1000 kPa absolute. Frequently, the temperature of the liquid in decanter 114 is between about 25° C. and 100° C. In decanter 114, two liquid phases exist, a denser, ethylene dichloride-rich phase and a less dense, aqueous phase. Ethylene dichloride, at 20° C. has a density slightly over 1.25 grams per cubic centimeter, and thus can be phase-separated readily from the aqueous phase. Water has little solubility in the ethylene dichloride-rich phase. For instance, in a binary system of water and ethylene dichloride, about 3500 parts per million by mass of water are soluble in ethylene dichloride at 50° C. The conditions in the decanter are more complex than those in a binary system, and by maintaining a substantial aqueous phase at a low pH, the polarity of the aqueous phase is increased, tending to reduce the amount of water in the ethylene dichloride phase and tending to extract more polar compounds such as monoethylene glycol, hydrogen chloride and 2-chloroethanol, and any catalyst if present, into the aqueous phase. Decanter 114 can be of any suitable design and includes gravity decanters, centrifuges and tricanters, and vane or baffle-assisting phase separators.

The denser, ethylene dichloride-rich phase is withdrawn from decanter 114 via line 116 and passed to washing unit operation 118 where it is contacted with at least a portion of the make-up monoethylene glycol supplied via line 120. The monoethylene glycol, which has a density of about 1.1 grams per cubic centimeter, is provided as a separate liquid phase from the ethylene dichloride and serves to extract further water, hydrogen chloride, and 2-chloroethanol from the ethylene dichloride. Any suitable washing unit operation can be used. Preferably, the washing unit operation provides for high shear contact to facilitate the extraction of impurities. The washing is conducted at a temperature and pressure below the boiling point of the ethylene dichloride under the pressure conditions. Preferably, the monoethylene glycol phase from the washing unit operation is used to cool the effluent from reactor 106, the temperature of the monoethylene glycol fed to the washing unit operation is less than about 35°° C., say, between about 0°° C. and 30° C. and the pressure is below about 500 kPa absolute. The washed ethylene dichloride phase sometimes has a water content less than about 5000, preferably less than about 2500, ppm by mass; a hydrogen chloride content of less than about 100 ppm by mass; a 2-chloroethanol content of less than about 100, preferably less than about 20, ppm by mass; and monoethylene glycol.

A less dense, monoethylene glycol phase is withdrawn from washing unit operation 118 via line 122. A washed, ethylene dichloride liquid phase is withdrawn from washing unit operation 118 via line 124 and is passed to distillation column 126. Distillation column 126 separates ethylene dichloride from monoethylene glycol that is dissolved in and entrained in the ethylene dichloride phase from washing unit operation 118. Any suitable distillation apparatus can be used. In instances where sufficient water is present to form an azeotrope of ethylene dichloride and water can be formed, an azeotropic distillation can be used. Typically, however, insufficient water is present to form azeotropes with ethylene dichloride, 2-chloroethanol or hydrogen chloride. An ethylene dichloride overhead is withdrawn via line 128 from distillation column 126 as the product. Often, the product contains at least 99.5, say at least 99.8, mass percent ethylene dichloride. With a normal boiling point of about 84° C., ethylene dichloride is facilely separated by distillation from 2-chloroethanol which has a normal boiling point of about 128° C. and monoethylene glycol having a normal boiling point of about 197° C. The bottoms fraction will also contain water.

The bottoms fraction from column 126 is withdrawn via line 130 and is shown as being passed via line 130 to reactor 106. Alternatively, the bottoms fraction can be combined with the monoethylene glycol phase from washing unit operation 118.

Returning to line 122 from washing unit operation 118, the separated monoethylene glycol phase is passed to heat exchanger 110 where it is used in indirect heat exchange to cool the reaction product from reactor 106. It is to be understood that additional heat exchangers may be required to cool the reaction product being passed to decanter 124 to the desired temperatures. The monoethylene glycol phase is heated as a result of the heat exchange and is passed to reactor 106 via lines 132 and 134.

Returning to decanter 114, the aqueous liquid phase exits via line 136. None, or a portion, of the aqueous phase can by-pass dehydration column 138 via line 140 and then line 134 to reactor 106. All, or the balance, of the aqueous phase is passed via line 136 to dehydration column 138. Dehydration column 138 is adapted for an azeotropic distillation to remove water. Preferably, the azeotroping agent provides an azeotrope having a normal boiling point of less than about 85° C., more preferably less than about 80° C., and is substantially immiscible with water. Examples include, but are not limited to, benzene, toluene, cyclohexane, methylene chloride, ethylene chloride, and propylene chloride. It is to be understood that in the presence of water, other azeotropes can form including that of 2-chloroethanol and water (normal boiling point 98° C.) and hydrogen chloride (normal boiling point 108° C.). Consequently, the 2-chloroethanol and hydrogen chloride will be in the bottoms fraction of column 138, preferably at least about 98 percent of each of the 2-chloroethanol and hydrogen chloride fed to column 138 will be in the bottoms fraction. The bottoms fraction exits via line 142 and is passed to reactor 106 via line 134.

The overhead fraction from dehydration column is withdrawn via line 144, condensed and passed to decanter 146. A denser, aqueous phase is withdrawn via line 148, and the less dense phase containing the azeotroping agent is recycled to column 138 via line 150.

The portion of the aqueous phase from decanter 134 that is passed to dehydration column 138 is sufficient to maintain a steady-state operation with a given fraction of water in the reaction product.

What is claimed is:

1. A process for making ethylene dichloride from monoethylene glycol and hydrochloric acid in the presence of water comprising:
a) contacting in a reaction zone monoethylene glycol and hydrochloric acid a temperature and for a time sufficient to convert at least 25 percent of the monoethylene glycol to ethylene dichloride, to provide a reaction product comprising a liquid phase product containing ethylene dichloride, water, hydrochloric acid, and 2-choroethanol and optionally a gas phase comprising azeotrope of ethylene dichloride and water, wherein
i. the temperature and pressure of the reaction is sufficient to provide a ratio of 2-chloroethanol in azeotrope with water in the gas phase to 2-chloroethanol in the liquid phase of less than 1:10,
ii. sufficient hydrogen chloride is present such that the pH of the reaction product is at a pH of below about 2, and
iii. the reaction product comprises between about 20 and 80 mass percent water;
b) providing the reaction product (i) at a temperature and pressure sufficient to provide a ratio of ethylene dichloride as an azeotropic mixture with water in the gas phase to ethylene dichloride in liquid phase of less than 1:10, and (ii) under conditions sufficient to form a denser, liquid phase rich in ethylene dichloride and a less dense liquid aqueous phase comprising water, hydrochloric acid, and 2-chloroethanol; and
c) selectively withdrawing from step (b) at least a portion of the heavier, liquid phase as an ethylene dichloride product.

2. The process of claim 1 wherein at the temperature and pressure of step (a) a gas phase comprising azeotrope of ethylene dichloride and water is formed.

3. The process of claim 2 wherein at least a portion of the gas phase is separately removed from the reaction zone of claim 1, condensed and passed to step (b).

4. The process of claim 2 wherein the reaction product is a mixed gas phase and liquid phase reaction product.

5. The process of claim 1 wherein step (a) is at a temperature and pressure sufficient to provide a ratio of ethylene dichloride as an azeotropic mixture with water in the gas phase to ethylene dichloride in liquid phase of less than 1:10.

6. The process of claim 1 wherein step (b) is conducted at a temperature below the boiling point at the pressure of step (b) of the ethylene dichloride and water azeotrope.

7. The process of claim 1 wherein the ethylene dichloride product of step (c) is contacted with substantially anhydrous monoethylene glycol whereby residual water, 2-chloroethanol and hydrochloric acid contained in the ethylene dichloride is stripped to the monoethylene glycol which is a less dense liquid phase than the denser, ethylene dichloride phase, and at least an aliquot portion is selectively separated from the ethylene dichloride phase to provide a purified ethylene dichloride product.

8. The process of claim 13 wherein the monoethylene glycol phase is passed to the reaction zone of step (a).

9. The process of claim 14 wherein the monoethylene glycol phase is used for indirect heat exchange with the reaction product passing to the decanting zone.

10. The process of claim 1 wherein at least a portion of the water in step (a) is from the reaction of hydrochloric acid with monoethylene glycol.

11. The process of claim 1 wherein steps (a), (b) and (c) are conducted on a continuous basis.

12. The process of claim 9 wherein at least an aliquot portion of the less dense liquid phase of step (b) is withdrawn from the decanting zone and is subjected to azeotropic distillation to provide an overhead comprising water and a bottoms fraction comprising 2-chloroethanol and hydrochloric acid, and at least a portion of the bottoms fraction is passed to the reaction zone.

13. The process of claim 12 wherein the azeotroping agent has a normal boiling point with water of less than about 85° C.

14. The process of claim 1 wherein the hydrochloric acid of step (a) is an aqueous hydrochloric acid.

15. The process of claim 14 wherein the aqueous hydrochloric acid contains 30 to 50 mass percent water.

16. The process of claim 1 wherein the hydrochloric acid of step (a) is anhydrous hydrochloric acid.

17. The process of claim 1 wherein catalyst is provided in the reaction zone of step (a)

18. The process of claim 17 wherein the catalyst comprises acetic acid.
